(19) United States
(12) Patent Application Publication
(10) Pub. No.: US 2021/0236054 A1
(43) Pub. Date: Aug. 5, 2021

(54) CHIROPRACTIC TREATMENT

(71) Applicants: Katinka van der Merwe, Fayetteville, AR (US); Manuel Mazzini, Fayetteville, AR (US)

(72) Inventors: Katinka van der Merwe, Fayetteville, AR (US); Manuel Mazzini, Fayetteville, AR (US)

(21) Appl. No.: 17/159,637

(22) Filed: Jan. 27, 2021

Related U.S. Application Data
(60) Provisional application No. 62/970,658, filed on Feb. 5, 2020.

(51) Int. Cl. A61B 5/00

(52) U.S. Cl. CPC A61B 5/4836; A61B 5/0053; A61B 5/4848; A61B 5/4561; A61B 5/4023

(57) ABSTRACT

A method for treating a patient suffering from a neuromuscular discomfort, including fibromyalgia, by categorizing the patient into one of several treatment categories, and treating the patient though novel sequences of adjustments including atlas adjustments and dura mater fixing.

### CROSS-REFERENCE TO RELATED APPLICATIONS

This application claims the benefit of priority of co-pending U.S. Provisional Application No. 62/970,658 filed on Feb. 5, 2020, which is herein incorporated by reference in its entirety.

### FIELD

The disclosed method relates generally to methods for treating patients by administration of a predetermined sequence of physical manipulations to the patient's body. More specifically, the disclosed provides resolution of neurological symptoms by manipulating the cranio-facio-atlanto-cervical region of a patient.

### BACKGROUND

Chiropractic treatment offers relief to patients suffering pain and other spino-neurologic symptoms through the employment of several different cranio-spinal adjustive procedures to remove nerve interference and effect improved nerve conductance and blood and cerebral spinal fluid flow. While most chiropractors use a diversified full spine approach, many employ one or more of Palmer upper cervical technique, Logan basic technique, cranial adjusting, a sacro-occipital technique, the Thompson Terminal Point Technique, the Meric System, an orthogonal atlas technique, the Neurologic Relief Centers Technique™ (NRCT), and the like.

Despite many chiropractic treatments, there still exists a need for reliable and effective chiropractic treatments for difficult-to-treat neurological disorders such as fibromyalgia, chronic fatigue, neuralgia, pain, and the like.

### SUMMARY

In one aspect the invention provides a method of treating a patient in need comprising (a) stimulating the vagus nerve of a patient by manipulating the atlas of the patient and (b) testing the sympathetic chain of the patient.

In one embodiment, the step of stimulating the vagus nerve (a) involves (i) positioning the atlas relative to the long axis of the spine at a 45 degree angle bilaterally anterior-to-posterior (A-P) and lateral-to-medial (L-M), or posterior-to-anterior (P-A) and L-M, and followed (ii) by a physical adjustment done by hand at line of drive of A-P and superior-to-inferior (S-I), or P-A and S-I.

In one embodiment, the step of testing the sympathetic chain (b) involves testing the strength of the patient's hamstring. In one embodiment, after bilaterally testing the patient's hamstring, (c-i) the practitioner adjusts the atlas or vertebra(e) at L5 or above. In another embodiment, after testing the patient's hamstrings, (c-ii) the practitioner adjusts the patient's atlas by pushing on patient's forehead with one hand while holding occiput in the other hand. In yet another embodiment, after testing the patient's hamstrings, (c-iii) the practitioner adjusts the patient's dura mater by spreading occipital-atlas-axis spinous processes while anchoring coccyx and sacrum.

In a second aspect, the invention provides a method of treating a patient in need comprising (a) testing the patient, followed by (b) stimulating the vagus nerve of a patient by manipulating the atlas of the patient, and followed by (c) testing the sympathetic chain of the patient.

In one embodiment, the step of testing the patient (a) involves (i) checking the posture of the patient from lateral and posterior views. In another embodiment, the step of testing the patient (a) involves (ii) testing the balance of the patient. In another embodiment, the step of testing the patient (a) involves (iii) the practitioner performing primary reflex tests on the patient. In another embodiment, the step of testing the patient (a) involves (iv) the practitioner performing neurological tests on the patient. In another embodiment, the step of testing the patient (a) involves (v) the practioners performing brain hemispheric tests on the patient. In yet another embodiment, the step of testing the patient (a) involves (i) checking the posture of the patient from lateral and posterior views, (ii) testing the balance of the patient, (iii) the practitioner performing primary reflex tests on the patient, (iv) the practitioner performing neurological tests on the patient, and (v) the practitioner performing brain hemispheric tests on the patient.

In one embodiment, the step of stimulating the vagus nerve (b) involves (i) positioning the atlas relative to the long axis of the spine at a 45 degree angle bilaterally anterior-to-posterior (A-P) and lateral-to-medial (L-M), or posterior-to-anterior (P-A) and L-M, and followed (ii) by a physical adjustment done by hand at line of drive of A-P and superior-to-inferior (S-I), or P-A and S-I.

In one embodiment, the step of testing the sympathetic chain (c) involves testing the strength of the patient's hamstring. In one embodiment, after bilaterally testing the patient's hamstring, (d-i) the practitioner adjusts the atlas or vertebra at L5 or above. In another embodiment, after testing the patient's hamstrings, (d-ii) the practitioner adjusts the patient's atlas by pushing on patient's forehead with one hand while holding occiput in the other hand. In yet another embodiment, after testing the patient's hamstrings, (d-iii) the practitioner adjusts the patient's dura mater by spreading occipital-atlas-axis spinous processes while anchoring coccyx and sacrum.

In a third aspect, the invention provides a method of treating a patient in need comprising (a) identifying patient by type, followed by (b) treating the patient according to type category. In one embodiment, the patient is identified as a type in need of dura matter fixing and treated according to the steps of (a) adjusting atlas, (b) fixing the dura mater, (c) tuning fork stimulation, (d) administering essential oils, and (e) percussive tapping brain.

In a fourth aspect, the invention provides a method for treating fibromyalgia, a neuralgia, pain, or a like malady by administering a method to a patient in need thereof according to any one of the first through fourth aspects.

### DRAWINGS

FIG. 1 is a flow chart depicting treatment steps for each of four types of patients.

FIG. 2 is a flow chart depicting patient testing steps.

FIG. 3 is a flow chart depicting steps for treating neuralgia or fibromyalgia.

FIG. 4 is a flow chart depicting treatment steps for each of four types of patients.

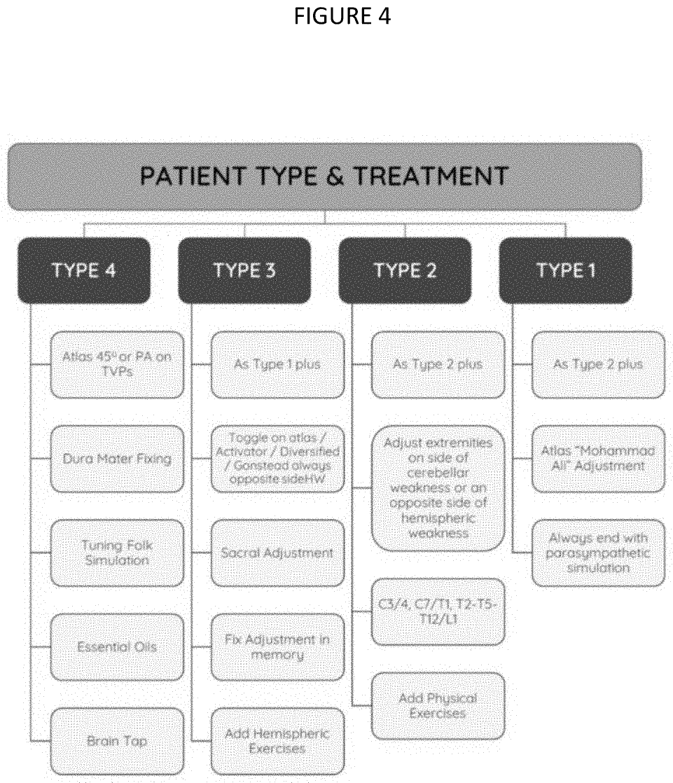

FIG. 5 is a flow chart depicting patient testing steps.

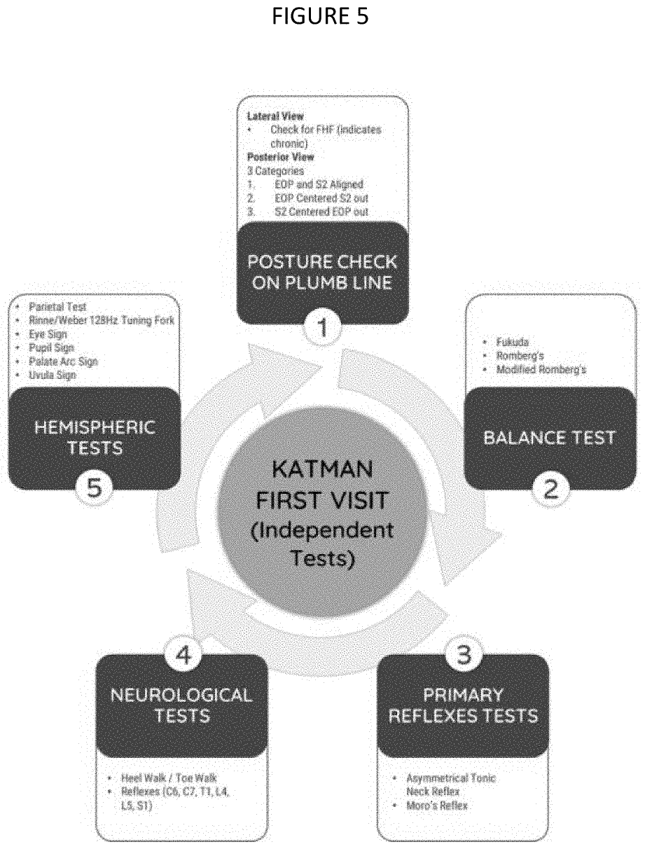

FIG. 6 is a flow chart depicting steps for treating neuralgia or fibromyalgia.

### DETAILED DESCRIPTION

Embodiments of the present invention involve the application of physical adjustments to a patient's head and neck using manual force or with assistance of mechanical equipment for the purpose of treating pain, fibromyalgia, and other neurological symptoms by adjusting the dura mater and atlas/axis/occipital-associated nerve processes. Some alternative embodiments of the present invention may utilize such instruments as an activator instrument, a toggle board, and a chiropractic table, all of which are described below.

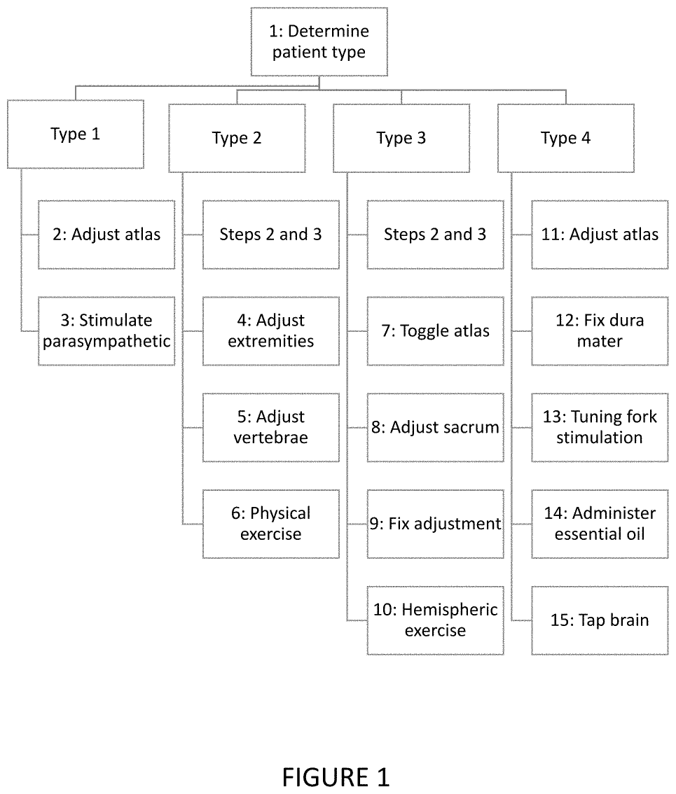

Referring to FIG. 1, disclosed is a method for treating a patient with pain, other neuralgia, fibromyalgia, or other neuromuscular discomfort. In one embodiment, the chiropractic practitioner interviews or tests the patient to determine the proper course of treatment for the particular type of patient (step 1). If the patient is determined to be a Type 1 patient, then the practioners adjusts the atlas of the patient (step 2). In one embodiment, the atlas is adjusted using the Muhammad Ali method. In another embodiment, the atlas is adjusted (step 2) by placing the patient in a supine position (step 2a), placing the patient's head on the practitioner's thenar processes (step 2b), practitioner applies a light touch to the lateral masses of the patient's atlas at a 45-degree angle anterior-to-posterior (A-P) and superior-to-inferior (S-I) bilaterally following the angle of the facets using the practitioner's middle fingers (step 2c), practitioner requests that the patient relax and to try not to help (step 2d), practitioner applies P-A and S-I pressure at a 45-degree angle on the posterior arch of the atlas bilaterally (step 2e1) or practioners applies A-P and S-I pressure at a 45-degree angle on the posterior arch of the atlas bilaterally (step 2e2).

In one embodiment, while the contacts of step 2 are being held, the patient's parasympathetic nervous system is stimulated (step 3). In one embodiment, the parasympathetic nervous system is stimulated by the practitioner requesting the patient and the patient complying to turn their eyes first and only to the right and then to the left respectively for about 1-1.5 minute each direction (step 3a). Concurrently with step 3a, the practitioner applies pressure to two points lateral to the spine (step 3b). In one embodiment, the two pressure points lateral to the spine are along the sympathetic chain, and the pressure is applied by inserting pressure balls at any two specific levels along the spine while the patient is in a supine position. In one particular embodiment, the two specific levels are left T4 and L4. In one embodiment, the practitioner verifies the direction of movement and sympathetic chain contact points by asking the patient how their feet feel, assessing the tone and position of the patient's feet, determining leg length, or muscle-testing the patient's feet while the patient is in the supine position (step 3c).

In another embodiment, if the patient is determined to be a Type 2 patient, steps 2 and 3 are performed as described for the Type 1 patient. After performing steps 2 and 3, the practitioner adjusts the patient's extremities on the side of the patient that displays cerebellar weakness or on the opposite side of the patient showing hemispheric weakness (step 4), followed by adjusting C3/4, C7/T1, T2-T5, and or T12/L1 (step 5), and optionally followed by physical exercise (step 6).

In another embodiment, if the patient is determined to be a Type 3 patient, steps 2 and 3 are performed as described for the Type 1 patient. After performing step 3, the practitioner adjusts the patient's atlas (step 7) using the toggle recoil technique (step 7a) followed in some embodiments by performing a Gonstead adjustment on the side of the patient showing hemispheric weakness (step 7b). After atlas adjustment (step 7), the practitioner adjusts the patient's sacrum (step 8), followed by fixing the adjustment (step 9), followed with hemispheric exercises (step 10).

Referring again to FIG. 1, in another embodiment, if the patient is determined to be a Type 4 patient, the practitioner adjusts the patient's atlas (step 11). In one embodiment, the atlas is adjusted (step 11) by placing the patient in a supine position (step 11a), placing the patient's head on the practitioner's thenar processes (step 11b), practitioner applies a light touch to the lateral masses of the patient's atlas at a 45-degree angle anterior-to-posterior (A-P) and superior-to-inferior (S-I) bilaterally following the angle of the facets using the practitioner's middle fingers (step 11c), practitioner requests that the patient relax and to try not to help (step 11d), practitioner applies P-A and S-I pressure at a 45-degree angle on the posterior arch of the atlas bilaterally (step 11e1) or practioners applies A-P and S-I pressure at a 45-degree angle on the posterior arch of the atlas bilaterally (step 11e2).

In one embodiment, after atlas adjusting (step 11), the dura mater of the patient is fixed by adjusting transverse processes associated with the occipital, atlas, and/or axis (i.e., EOP-Atlas-Axis or EOP-A-A) (step 12).

In one embodiment, after fixing the dura mater (step 12), the practitioner administers tuning fork stimulation to select vertebrae or EOP-A-A (step 13).

In one embodiment, after tuning fork stimulation (step 13), essential oil is administered to the patient (step 14). In one embodiment, the essential oil is administered to the side of the patient that exhibits brain weakness by deep inhalation, such as, e.g., by inhaling deeply three times. In one embodiment, lemon essential oil is administered to the left nostril and lavender essential oil is administered to the right nostril.

In one embodiment, after administering essential oil (step 14), the practitoner administers neuro-algorithms to the patient to produce brainwave entrainment, also known as BRAINTAP (Braintap Technologies, New Bern, N.C.) (step 15). The neuro-algorithms include tonal therapy, guided visualization, and 10-cycle holographic music, usually administered through a headset and visor system.

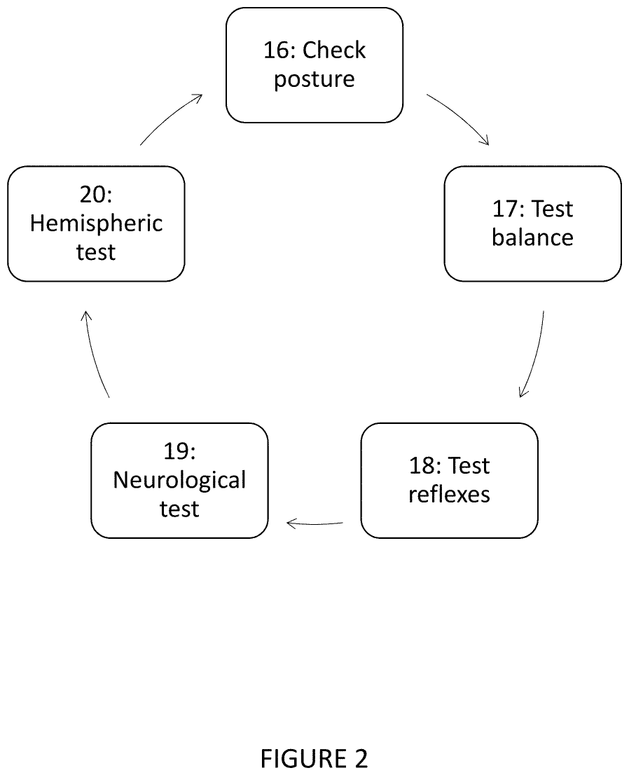

Referring to FIG. 2, in one embodiment the practitioner performs a series of tests on a patient generally at the first visit. In one embodiment, the posture of the patient is checked (step 16). In one specific embodiment, the posture is checked on a plumb line with a lateral view checking for FHF, and a posterior view checking for alignment of EOP and S2. The patient is then categorized as (i) EOP and S2 aligned, (ii) EOP centered and S2 out, or (iii) S2 centered.

In one embodiment, the balance of the patient is checked (step 17). In one specific embodiment, the balance is tested using the method of Fukuda stepping or Unterberger step test test for vestibular function (see Fukuda, T. (1959), “The stepping test: two phases of the labyrinthine reflex,” Acta Otolaryngol 50(2): 95-108). In another specific embodiment, patient balance is tested using the Romberg method of modified Romberg method (see Reicke, N, “The Romberg head-shake test within the scope of equilibrium diagnosis,” H.N.O 40, 195-201 (1992); and Agrawal et al., “The modified Romberg balance test: normative data in US adults,” Otology & Neurotology, 2011 October, 32(8): 1309-1311).

In one embodiment, the patient's reflexes are checked (step 18). In one specific embodiment, the reflexes are tested via asymmetrical tonic neck reflex (ATNR) (see Parmenter, “An asymmetrical tonic neck reflex rating scale,” Am. J. Occup. Ther. 1983 July, 37(7): 462-465). In another specific embodiment, the patient's reflexes are tested via Moro's reflex (see Lehman R K, Schor N F. Neurologic evaluation. In: Kliegman R M, Stanton B F, St. Geme J W, Schor N F, eds. Nelson Textbook of Pediatrics. 20 th ed. Philadelphia, Pa.: Elsevier; 2016:chap 590).

In one embodiment, the practitioner performs neurological testing on the patient (step 19). In one specific embodiment, the neurological test involves heel walk and toe walk testing. In another specific embodiment, the neurological testing involves measuring reflexes associated with C6, C7, T1, L4, L5, and S1. In some embodiments, the neurological testing involves both heel walk/toe walk and vertebral reflex testing.

In one embodiment, the practitioner performs brain hemispheric testing on the patient (step 20) (see generally https://chiro.org/ACAPress/Orthopedic and Neurologic Procedures.html# Tests). In one specific embodiment, the hemispheric testing includes any one or more of the parietal test, Rinne and Weber test, eye sign, pupil sign, palate arc sign, and uvula sign tests.

In one embodiment, the patient is tested according to any one or more of steps 16-20 performed in any order.

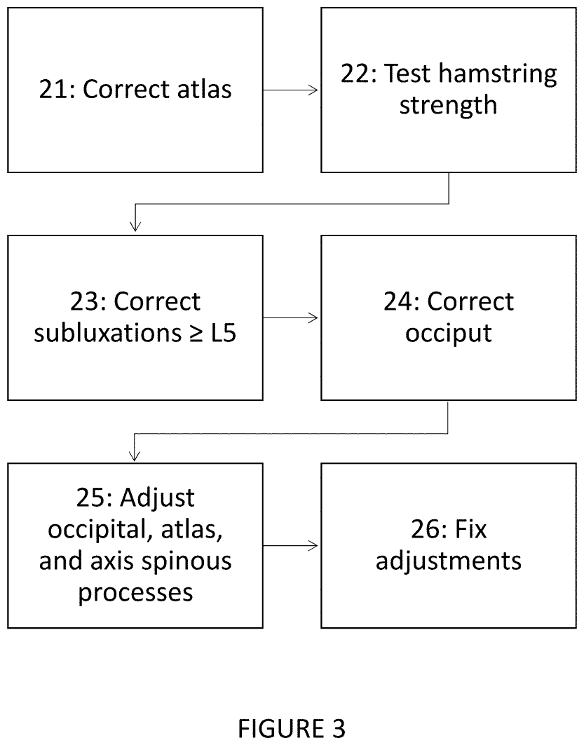

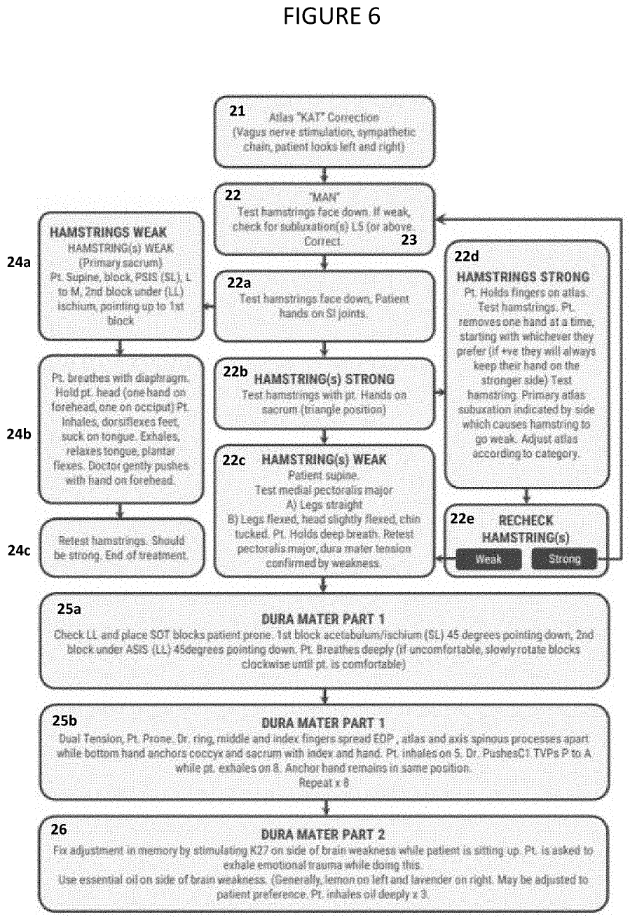

Referring to FIG. 3 and FIG. 6, in one embodiment, a patient with pain, fibromyalgia, neuralgia, neurological symptoms, or similar ailments is treated by (a) manipulation of the atlas (step 21), followed by (b) testing hamstring strength (step 22), followed by (c) correcting subluxations at L5 and above if the hamstring is/are weak (step 23), and followed by (d) manipulation of the occipital (step 24), or alternatively (e) manipulation/adjustment of the spinous processes of the occipital, atlas, and/or axis (step 25), and followed by (f) fixing the adjustment (step 26).

In a specific embodiment, step 21 involves stimulating the vagus nerve, contacting the sympathetic chain, and testing, and testing or determining the contacts by having the patient look in one direction (left or right) and then the other direction (right or left). In a specific embodiment of step 21, the atlas is moved with a 45-degree angle bilaterally A-P and L-M, or P-A and L-M, followed by a physical adjustment done by hand. In another specific embodiment of step 21, the atlas is adjusted by employing a line of drive that is A-P and S-I, or P-A and S-I.

In a specific embodiment, step 22 involves testing the hamstring face down. If the hamstrings are weak, then subluxations are checked at L5 and above and corrected if needed (step 22). In another embodiment, the hamstrings are tested with patient's hands on their sacroiliac joints (step 22a). If the hamstrings are initially determined to be strong, then the hamstrings are tested again with the patient's hands on their sacrum in a triangle position (step 22b). If the retested hamstrings at step 22b are then determined to be weak, then the patient is placed in a supine position and the medial pectoralis major is tested by starting patient with straight legs, then flexed legs with head slightly flexed and chin tucked, wherein the patient takes a deep breath and holds (step 22c). The pectoralis is retested and, if weak, then tension in the dura mater is confirmed.

If the hamstrings are determined to be weak at step 22a, then occiput correction is performed on the patient (step 24). In one embodiment, occiput correction (step 24) includes the steps of (a) placing the patient in a supine position with a first block placed at posterior superior iliac spine (SL) lateral to medial and a second block placed under lower lumbar at ischium pointing up to the first block (step 24a), (b) patient breathes with diaphragm, (c) practitioner places one hand on patient's forehead and the other hand on the patient's occiput, (d) patient inhales, dorsiflexes their feet and sucks on their tongue, (e) patient exhales, relaxes tongue and plantar flexes, and (f) the practitioner gently pushes with hand on patient's forehead (step 24b). The hamstrings are retested, and if strong, then the treatment session is ended (step 24c).

If the hamstrings are determined to be strong at step 22b, then the hamstrings are retested as follows: (a) the patient, while face down, holds their fingers on their atlas, (b) the hamstrings are tested, (c) patient removes one hand at a time starting with whichever they prefer (usually patient's keeps hand on stronger side), (d) practitioner tests hamstring, which indicates a primary atlas subluxation according to which side causes the hamstring to go weak, (e) practitioner adjusts the atlas according to the results at (d). The hamstring is then rechecked (step 22e). If the hamstring(s) is/are weak, then the practitioner performs step 22c.

In one embodiment, if tension in the dura mater is confirmed at step 22c, then the practitioner adjusts the patient's dura mater (step 25). In one embodiment, the dura mater is adjusted by (a) checking the lower lumber, (b) placing sacro occipital technique blocks under prone patient, wherein the first block is placed at acetabulum/ischium pointing down at 45-degrees and the second block placed at anterior superior iliac spine pointing down at 45-degrees, (c) the patient breathes deeply, and if the patient is uncomfortable, the practitioner slowly rotates the blocks clockwise until the patient is comfortable (step 25a). Then, in one embodiment, (d) while the patient is prone, the practitioner spreads external occipital protuberance (EOP)-atlas-axis spinous processes apart with their ring, middle, and index fingers while the practitioner's bottom hand anchors the coccyx and sacrum with index finger and hand, (e) the patient inhales on the count of five, (f) the practitioner pushes C1 transverse processes posterior to anterior while the patient exhales on the count of eight and the practitioner's anchor hand remains in place. Sub-steps (d) and (e) are repeat eight times (step 25b).

In one embodiment, after adjusting the dura mater (step 25), the adjustment is fixed into memory (step 26). In one embodiment, the adjustment is fixed into memory by stimulating the K27 point (acupressure point below clavicle near sternum) on patient's side of brain weakness while patient is sitting up (step 26a). The patient then exhales emotional trauma while K27 is stimulated (step 26b). The patient then deeply inhales essential oil at least three times on the side of brain weakness this (step 26c). In one embodiment, lemon essential oil is administered on the left and lavender essential oil is administered on the right. In another embodiment, the patient choses which oil to inhale.

In one embodiment, a patient with pain, neuralgia, fibromyalgia or the like is treated according to the steps set forth in FIG. 6, which adjusts the pia mater to enable improved nerve conduction and cerebral spinal fluid flow.

1. A method of treating fibromyalgia in a patient in need thereof, the method comprising:
manipulating the atlas of the patient to stimulate the vagus nerve; and
testing the sympathetic chain of the patient.

2. The method of claim 1, wherein the step of manipulating the atlas of the patient comprises:
positioning the atlas relative to the long axis of the spine at a 45 degree angle bilaterally anterior-to-posterior (A-P) and lateral-to-medial (L-M), or posterior-to-anterior (P-A) and L-M; and
adjusting by hand at line of drive of A-P and superior-to-inferior (S-I), or P-A and S-I.

3. The method of claim 1, wherein the step of testing the sympathetic chain comprises testing the strength of the patient's hamstring.

4. The method of claim 1 further comprising after the testing of the sympathetic chain of the patient:
adjusting the atlas or vertebra(e) at L5 or above;
adjusting the atlas by pushing on patient's forehead with one hand while holding patient's occiput in the other hand;
testing the patient's hamstring; and
adjusting the patient's dura mater by spreading occipital-atlas-axis spinous processes while anchoring patient's coccyx and sacrum.

5. The method of claim 1 further comprising testing the patient before manipulating the atlas of the patient, said testing comprising any one or more of:
a. checking the posture of the patient from lateral and posterior views;
b. testing the balance of the patient. In another embodiment;
c. performing primary reflex tests on the patient;
d. performing neurological tests on the patient; and
e. performing brain hemispheric tests on the patient.

6. A method of treating a neuromuscular discomfort in a patient in need thereof, the method comprising:
a. identifying the patient as any one of a type selected from the group consisting of type 1, type 2, type 3, and type 4;
b. treating the patient according to the identified type.

7. The method of claim 6, wherein a type 1 patient is treated by:
adjusting the patient's atlas; and
stimulating the patent's parasympathetic.

8. The method of claim 6, wherein a type 2 patient is treated by:
adjusting the patient's atlas;
stimulating the patient's parasympathetic;
adjusting the patient's extremities;
adjusting the patient's vertebrae; and
having the patient perform physical exercise.

9. The method of claim 6, wherein a type 3patient is treated by:
adjusting the patient's atlas;
stimulating the patient's parasympathetic;
toggling the patient's atlas;
adjusting the patient's sacrum;
fixing the adjustment in memory; and
having the patient perform hemispheric exercises.

10. The method of claim 6, wherein a type 4 patient is treated by:
adjusting the patient's atlas;
fixing the patient's dura mater;
stimulating the patient with a tuning fork;
administering an essential oil; and
percussive-tapping the patient's brain.

11. The method of claim 6, wherein the neuromuscular discomfort is selected from the group consisting of fibromyalgia, chronic fatigue, neuralgia, and pain.

12. The method of claim 6, wherein the step of identifying the patient as any one of a type selected from the group consisting of type 1, type 2, type 3, and type 4 comprises:
checking the patient's posture;
testing the patient's balance;
testing the patient's reflexes;
performing a neurological test on the patient; and
performing a hemispheric test on the patient.

13. The method of claim 12, wherein the step of checking the patient's posture comprises performing a posture check on a plumb line from a lateral view and a posterior view.

14. The method of claim 12, wherein the step of testing the patient's balance comprises one or more of a Fukuda test, Romberg's test, and modified Romberg's test.

15. The method of claim 12, wherein step of testing the patient's reflexes comprises performing an asymmetrical tonic neck reflex test and a Moro's reflex test.

16. The method of claim 12,wherein the neurological test comprises one or more of a heel walk/toe walk test, and reflex tests for C6, C7, T1, L4, L5, and S1.

17. The method of claim 12, wherein the hemispheric test comprises one or more of a parietal test, a Rinne/Weber 128 Hz tuning fork test, an eye sign test, a pupil sign test, a palate arc sign test, and a uvula sign test.

18. A method of treating a neuromuscular discomfort in a patient in need thereof, the method comprising:
a. correcting the patient's atlas;
b. testing the patient's hamstring;
c. correcting subluxations at patient's L5 vertebra;
d. correcting the patient's occiput;
e. adjusting the patient's occipital, atlas, and axis spinous processes; and
f. fixing the adjustments.

19. The method of claim 18, wherein the wherein the neuromuscular discomfort is selected from the group consisting of fibromyalgia, chronic fatigue, neuralgia, and pain.

20. The method of claim 19, wherein correcting the patient's atlas involves the patient looking left and right, and stimulates the vagus nerve and sympathetic chain.
